(19) United States
(12) Patent Application Publication
(10) Pub. No.: US 2008/0003271 A1
(43) Pub. Date: Jan. 3, 2008

(54) Compositions with Several Hyaluronic Acid Fractions for Cosmetic Use

(75) Inventors: Khadija Schwach Abdellaoui, Frederiksberg C (DK); Birgitte Molholm Malle, Birkerod (DK)

Correspondence Address: NOVOZYMES NORTH AMERICA, INC., 500 FIFTH AVENUE, SUITE 1600, NEW YORK, NY, 10110, US

(73) Assignee: Novozymes BioPolymer A/S, Bagsvaerd (DK)

(21) Appl. No.: 11/769,141

(22) Filed: Jun. 27, 2007

Related U.S. Application Data
(60) Provisional application No. 60/819,683, filed on Jul. 10, 2006.
(60) Provisional application No. 60/817,766, filed on Jun. 29, 2006.

(30) Foreign Application Priority Data
Jun. 28, 2006 (DK) PA 2006 00865
Jul. 7, 2006 (DK) PA 2006 00937

(51) Int. Cl. A61K 31/728; A61F 13/00; A61K 9/50; A61P 17/02; A61P 17/18; A61P 19/02; A61P 27/02; A61P 35/00

(52) U.S. Cl. 424/445; 424/448; 424/490; 514/62

(57) ABSTRACT

The present invention provides a moisturizing, cosmetic, or anti-wrinkle product comprising at least two hyaluronic acid fractions, or salts thereof, wherein a fraction has an average molecular weight in the range of 8,000-100,000 Da, and a fraction has an average molecular weight in the range of 100,000-500,000 Da.

### CROSS-REFERENCE TO RELATED APPLICATIONS

This application claims priority or the benefit under 35 U.S.C. 119 of Danish application nos. PA 2006 00865 and PA 2006 00937 filed Jun. 28, 2006 and Jul. 7, 2006, respectively, and U.S. provisional application Nos. 60/817,766 and 60/819,683 filed Jun. 29, 2006 and Jul. 10, 2006, respectively, the contents of which are fully incorporated herein by reference.

### FIELD OF THE INVENTION

The present invention relates to compositions comprising at least two hyaluronic acid (HA) fractions or salts thereof, a fraction of HA having a very low average molecular weight (MW) and a low-medium MW HA fraction, for use in moisturizing, cosmetic, or anti-wrinkle formulations to decrease both deep and superficial wrinkles.

### BACKGROUND OF THE INVENTION

The most abundant heteropolysaccharides of the body are the glycosaminoglycans. Glycosaminoglycans are unbranched carbohydrate polymers, consisting of repeating disaccharde units (only keratan sulphate is branched in the core region of the carbohydrate). The disaccharide units generally comprise, as a first saccharide unit, one of two modified sugars—N-acetylgalactosamine (GalNAc) or N-acetylglucosamine (GlcNAc). The second unit is usually an uronic acid, such as glucuronic acid (GlcUA) or iduronate.

Glycosaminoglycans are negatively charged molecules, and have an extended conformation that imparts high viscosity when in solution. Glycosaminoglycans are located primarily on the surface of cells or in the extracellular matrix. Glycosaminoglycans also have low compressibility in solution and, as a result, are ideal as a physiological lubricating fluid, e.g., joints. The rigidity of glycosaminoglycans provides structural integrity to cells and provides passageways between cells, allowing for cell migration. The glycosaminoglycans of highest physiological importance are hyaluronan, chondroitin sulfate, heparin, heparan sulfate, dermatan sulfate, and keratan sulfate. Most glycosaminoglycans bind covalently to a proteoglycan core protein through specific oligosaccharide structures. Hyaluronan forms large aggregates with certain proteoglycans, but is an exception as free carbohydrate chains form non-covalent complexes with proteoglycans.

Numerous roles of hyaluronan in the body have been identified (see, Laurent T. C. and Fraser J. R. E., 1992, FASEB J. 6: 2397-2404; and Toole B. P., 1991, “Proteoglycans and hyaluronan in morphogenesis and differentiation.” In: Cell Biology of the Extracellular Matrix, pp. 305-341, Hay E. D., ed., Plenum, New York). Hyaluronan is present in hyaline cartilage, synovial joint fluid, and skin tissue, both dermis and epidermis. Hyaluronan is also suspected of having a role in numerous physiological functions, such as adhesion, development, cell motility, cancer, angiogenesis, and wound healing. Due to the unique physical and biological properties of hyaluronan, it is employed in eye and joint surgery and is being evaluated in other medical procedures.

The term “hyaluronic acid” is used in literature to mean acidic polysaccharides with different molecular weights constituted by residues of D-glucuronic and N-acetyl-D-glucosamine acids, which occur naturally in cell surfaces, in the basic extracellular substances of the connective tissue of vertebrates, in the synovial fluid of the joints, in the endobulbar fluid of the eye, in human umbilical cord tissue and in cocks' combs.

The term “hyaluronic acid” is in fact usually used as meaning a whole series of polysaccharides with alternating residues of D-glucuronic and N-acetyl-D-glucosamine acids with varying molecular weights or even the degraded fractions of the same, and it would therefore seem more correct to use the plural term of “hyaluronic acids”. The singular term will, however, be used all the same in this description; in addition, the abbreviation “HA” will frequently be used in place of this collective term.

HA plays an important role in the biological organism, as a mechanical support for the cells of many tissues, such as the skin, tendons, muscles and cartilage, it is a main component of the intercellular matrix. HA also plays other important parts in the biological processes, such as the moistening of tissues, and lubrication.

HA may be extracted from the above mentioned natural tissues, although today it is preferred to prepare it by microbiological methods to minimize the potential risk of transferring infectious agents, and to increase product uniformity, quality and availability (WO 03/0175902, Novozymes).

HA and its various molecular size fractions and the respective salts thereof have been used as medicaments, especially in treatment of arthropathies, as an auxiliary and/or substitute agent for natural organs and tissues, especially in ophtalmology and cosmetic surgery, and as agents in cosmetic preparations. Products of hyaluronan have also been developed for use in orthopaedics, rheumatology, and dermatology.

High molecular weight fractions of HA having an average molecular weight of about 1 to about 1.5 MDa are well known for providing excellent moisturizing properties in cosmetic compositions such as lotions and creams.

Very tow molecular weight fractions of HA have been reported to exhibit anti-wrinkle properties, allegedly due to the ability of these fractions to penetrate the skin barrier.

Both moisturization and anti-wrinkle properties are highly desirable in many applications, and a single composition exhibiting both would be of great commercial interest.

### SUMMARY OF THE INVENTION

The present inventors recently formulated several HA-compositions comprising two separate HA fractions, one having a very low average molecular weight, and another fraction with a low-medium average molecular weight, and they evaluated these fractions for moisturizing and anti-wrinkle effects.

Surprisingly, it was found that these compositions exhibited both moisturizing as well as anti-wrinkle effects.

Accordingly, in a first aspect the invention relates to a moisturizing, cosmetic, or anti-wrinkle product comprising at least two hyaluronic acid fractions, or salts thereof, wherein a fraction has an average molecular weight in the range of 8,000-100,000 Da, preferably 10-90 kDa, or preferably 20-80 kDa, or 30-70 kDa, even more preferably in the range of 40-60 kDa, or most preferably about 50 kDa; and another fraction has an average molecular weight in the range of 100,000-500,000 Da, or preferably 150-450 kDa, more preferably 200-400 kDa, even more preferably in the range of 250-350 kDa, or most preferably around 300 kDa.

In a second aspect, the invention relates to a composition comprising a product as defined in the first aspect, and an active ingredient, preferably the active ingredient is a pharmacologically active agent.

A third aspect of the invention relates to a pharmaceutical composition comprising an effective amount of a product as defined in the first aspect, together with a pharmaceutically acceptable carrier, excipient or diluent.

A fourth aspect relates to a pharmaceutical composition comprising an effective amount of a product as defined in the first aspect as a vehicle, together with a pharmacologically active agent.

A fifth aspect relates to a cosmetic article comprising as an active ingredient an effective amount of a product as defined in the first aspect.

In a sixth aspect, the invention relates to a sanitary, medical or surgical article comprising a product as defined in the first aspect, preferably the article is a surgical sponge, a wound healing sponge, or a part comprised in a band aid or other wound dressing material.

An important aspect relates to a medicament capsule or microcapsule comprising a product as defined in the first aspect.

Final aspects of the invention relate to methods of performing procedures in ophtalmology, in the treatment of osteoarthritis or cancer, of treating a wound, of performing dermal or transdermal administration of a pharmacologically active agent, or dermal administration of a cosmetic, the improvement which comprises the use of a product as defined in the first aspect, or a composition as defined in any of the second, third, or fourth aspects.

A number of aspects relate to uses of a product as defined in the first aspect or a composition as defined in any of the preceding aspects, for the manufacture of a medicament for the treatment of osteoarthritis, cancer, the manufacture of a medicament for an ophtalmological treatment, the manufacture of a medicament for the treatment of a wound, the manufacture of a medicament for angiogenesis, or the manufacture of a moisturizer.

### BRIEF DESCRIPTION OF DRAWINGS

In some of the figures, the histograms are indicated with one asterisk, denoting a statistically significantly difference value from that at t=0; p≦0.05. Histograms indicated with a double asterisk are statistically highly significantly different value from that at t=0; p≦0.01.

FIG. 1: Comparative evaluation of the relative long term skin hydration. A significant increase in hydration was obtained with 3 molecular weight fractions of HA after 4 weeks and 8 weeks of treatment.

FIG. 2: Shows the various skin-elasticity parameters that are measured with a cutometer, as described in the detailed description below.

FIG. 3: Shows the relative measured overall skin elasticities, R2, after 4 weeks and 8 weeks of application. A significant increase of the overall elasticity (R2) was clearly observed with all active creams. No significant difference was observed between the different molecular weight HA fractions.

FIG. 4: The relative mean roughness measurements are described below and the results are shown in FIG. 4. The mean roughness values decreased significantly after 4 and 8 weeks of application. The effect was more pronounced for the 300,000 Da MW fraction which accumulates preferentially at the surface of the skin.

FIG. 5: The relative max roughness measurements are described below, and the results are shown in FIG. 5; these values also decreased significantly after 4 and 8 weeks of application, but only for the two lowest molecular weight fractions. This effect was significantly more pronounced for the very low molecular fraction HA of 50 kDa which is able to penetrate the skin.

FIG. 6: Shows the relative viscoelastic ratio, R6, after 4 and 8 weeks of application as described in the examples below. A highly significant increase was observed with the low MW HA fraction.

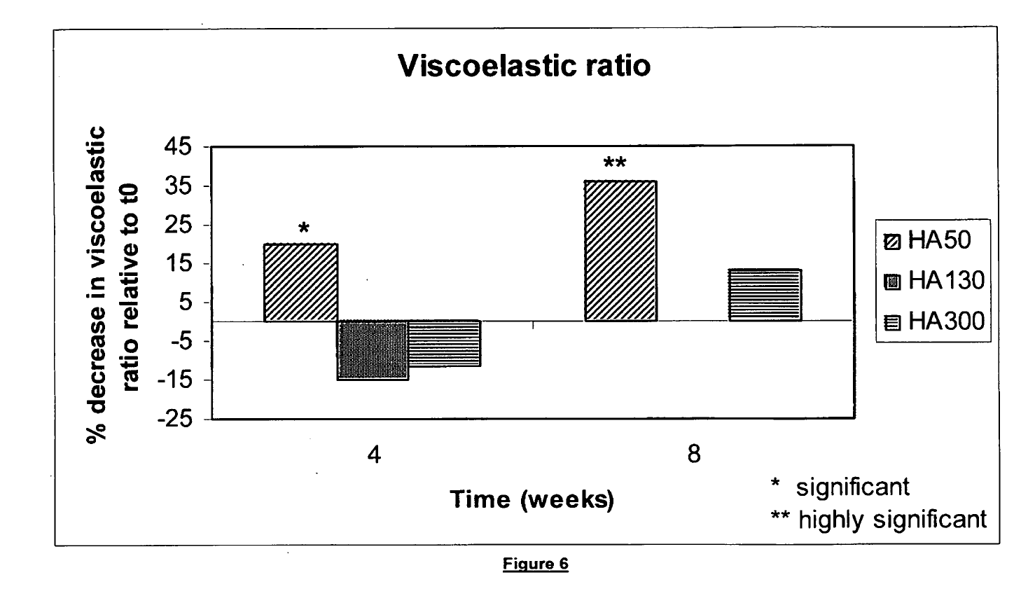

### DETAILED DESCRIPTION OF THE INVENTION

“Hyaluronic acid” is defined herein as an unsulphated glycosaminoglycan composed of repeating disaccharide units of N-acetylglucosamine (GlcNAc) and glucuronic acid (GlcUA) linked together by alternating beta-1,4 and beta-1,3 glycosidic bonds. Hyaluronic acid is also known as hyaluronan, hyaluronate, or HA. The terms hyaluronan and hyaluronic acid are used interchangeably herein.

A first aspect of the invention relates to a moisturizing, cosmetic, or anti-wrinkle product comprising at least two hyaluronic acid fractions, or salts thereof, wherein a fraction has an average molecular weight in the range of 8,000-100,000 Da, preferably 10-90 kDa, or preferably 20-80 kDa, or 30-70 kDa, even more preferably in the range of 40-60 kDa, or most preferably about 50 kDa; and another fraction has an average molecular weight in the range of 100,000-500,000 Da, or preferably 150-450 kDa, more preferably 200-400 kDa, even more preferably in the range of 250-350 kDa, or most preferably around 300 kDa.

Rooster combs are a significant commercial source for hyaluronan. Microorganisms are an alternative source. U.S. Pat. No. 4,801,539 discloses a fermentation method for preparing hyaluronic acid involving a strain of Streptococcus zooepidemicus with reported yields of about 3.6 g of hyaluronic acid per liter. European Patent No, EP 0694616 discloses fermentation processes using an improved strain of Streptococcus zooepidemicus with reported yields of about 3.5 g of hyaluronic acid per liter. As disclosed in WO 03/054163 (Novozymes), which is incorporated herein in its entirety, hyaluronic acid or salts thereof may be recombinantly produced, e.g., in a gram-positive Bacillus host.

Hyaluronan synthases have been described from vertebrates, bacterial pathogens, and algal viruses (DeAngelis, P. L., 1999, Cell. Mol. Life Sci. 56: 670-682). WO 99/23227 discloses a Group I hyaluronate synthase from Streptococcus equisimilis. WO 99/51265 and WO 00/27437 describe a Group II hyaluronate synthase from Pasturella multocida. Ferretti et al. disclose the hyaluronan synthase operon of Streptococcus pyogenes, which is composed of three genes, hasA, hasB, and hasC, that encode hyaluronate synthase, UDP glucose dehydrogenase, and UDP-glucose pyrophosphorylase, respectively (Proc. Natl. Acad. Sci. USA. 98, 4658-4663, 2001). WO 99/51265 describes a nucleic acid segment having a coding region for a Streptococcus equisimilis hyaluronan synthase.

Since the hyaluronan of a recombinant Bacillus cell is expressed directly to the culture medium, a simple process may be used to isolate the hyaluronan from the culture medium. First, the Bacillus cells and cellular debris are physically removed from the culture medium. The culture medium may be diluted first, if desired, to reduce the viscosity of the medium. Many methods are known to those skilled in the art for removing cells from culture medium, such as centrifugation or microfiltration. If desired, the remaining supernatant may then be filtered, such as by ultrafiltration, to concentrate and remove small molecule contaminants from the hyaluronan. Following removal of the cells and cellular debris, a simple precipitation of the hyaluronan from the medium is performed by known mechanisms. Salt, alcohol, or combinations of salt and alcohol may be used to precipitate the hyaluronan from the filtrate. Once reduced to a precipitate, the hyaluronan can be easily isolated from the solution by physical means. The hyaluronan may be dried or concentrated from the filtrate solution by using evaporative techniques known to the art, such as lyophilization or spraydrying.

Host Cells

A preferred embodiment relates to the product of the first aspect, wherein the hyaluronic acid or salt thereof is recombinantly produced, preferably by a Gram-positive bacterium or host cell, more preferably by a bacterium of the genus Bacillus.

The host cell may be any Bacillus cell suitable for recombinant production of hyaluronic acid. The Bacillus host cell may be a wild-type Bacillus cell or a mutant thereof. Bacillus cells useful in the practice of the present invention include, but are not limited to, Bacillus agaraderhens, Bacillus alkalophilus, Bacillus amyloliquefaciens, Bacillus brevis, Bacillus circulans, Bacillus clausii, Bacillus coagulans, Bacillus firmus, bacillus lautus, Bacillus lentus, Bacillus licheniformis, Bacillus megaterium, Bacillus pumilus, Bacillus stearothermophilus, Bacillus subtilis, and Bacillus thuringiensis cells. Mutant Bacillus subtilis cells particularly adapted for recombinant expression are described in WO 98/22598. Non-encapsulating Bacillus cells are particularly useful in the present invention.

In a preferred embodiment, the Bacillus host cell is a Bacillus amyloliquefaciens, Bacillus clausii, Bacillus lentus, Bacillus licheniformis, Bacillus stearothermophilus or Bacillus subtilis cell. In a more preferred embodiment, the Bacillus cell is a Bacillus amyloliquefaciens cell. In another more preferred embodiment, the Bacillus cell is a Bacillus clausii cell. In another more preferred embodiment, the Bacillus cell is a Bacillus lentus cell. In another more preferred embodiment, the Bacillus cell is a Bacillus licheniformis cell. In another more preferred embodiment, the Bacillus cell is a Bacillus subtilis cell. In a most preferred embodiment, the Bacillus host cell is Bacillus subtilis A164Δ5 (see U.S. Pat. No. 5,891,701) or Bacillus subtilis 168Δ4.

Transformation of the Bacillus host cell with a nucleic acid construct of the present invention may, for instance, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6; 742-751), or by conjugation (see, e.g., Koehler and Thorne, 1987, Journal of Bacteriology 169. 5271-5278).

Molecular Weight

The level of hyaluronic acid may be determined according to the modified carbazole method (Bitter and Muir, 1962, Anal Biochem. 4: 330-334). Moreover, the average molecular weight of the hyaluronic acid may be determined using standard methods in the art, such as those described by Ueno et al., 1988, Chem, Pharm. Bull. 36, 4971-4975; Wyatt, 1993, Anal. Chim. Acta 272: 1-40; and Wyatt Technologies, 1999, “Light Scattering University DAWN Course Manual” and “DAWN EOS Manual” Wyatt Technology Corporation, Santa Barbara, Calif.

Salts and Crosslinked HA

A preferred embodiment relates to a product of the first aspect, which comprises an inorganic salt of hyaluronic acid, preferably sodium hyaluronate, potassium hyaluronate, ammonium hyaluronate, calcium hyaluronate, magnesium hyaluronate, zinc hyaluronate, or cobalt hyaluronate.

It has been found that the reaction of sodium hyaluronate with polylactic acid mono or di-acyl chloride resulted in a linked or crosslinked HA-PLA or HA-PLA-HA product, which showed an intensified peak at 1736 cm-1 on the IR spectrum, when compared to a standard spectrum of the untreated HA or PLA, corresponding to the presence of newly formed polylactic esters in the linked HA-PLA product.

Accordingly, a preferred embodiment relates to the product of the first aspect, wherein the hyaluronic acid or salt thereof comprises esters of a polymeric alpha hydroxy acid, preferably of polylactic acid or glycolic acid.

It has also been found that treatment of a solution of sodium hyaluronate with boric acid resulted in a crossinked HA-borate hydrogel which showed new peaks at 1200 and 945 cm-1 on the FT-IR spectrum, when compared to a standard spectrum of the untreated Na-HA, corresponding to the presence of newly formed borate esters in the crosslinked HA-borate hydrogel.

Accordingly, a preferred embodiment relates to the product of the first aspect, wherein the hyaluronic acid or salt thereof comprises borate esters.

In another preferred embodiment of the product of the first aspect, the hyaluronic acid or salt thereof is fully or partially crosslinked with divinylsulfone (DVS).

Moisturizing and Anti-wrinkle Effects

As shown in the examples below, a product of the first aspect has a skin moisturizing effect, expressed as a capability of increasing the skin hydration value, which in a preferred embodiment is at least 3% over 8 weeks, preferably at least 5%, most preferably at least 7%, when measured as defined below in the examples.

Further, a product of the first aspect is capable of increasing overall skin elasticity, R2, which in a preferred embodiment is increased with at least 4% over 8 weeks, preferably at least 8%, more preferably at least 12%, when measured as defined below in the examples.

Also, in a preferred embodiment, the product of the first aspect is capable of reducing the mean roughness value of skin with at least 5% over 8 weeks, preferably at least 10%, and most preferably at least 15%, when measured as defined herein.

In yet another preferred embodiment, the product of the first aspect is capable of reducing the maximum roughness value of skin with at least 3% over 8 weeks, preferably at least 5%, and most preferably at least 10%, when measured as defined herein.

Another preferred embodiment of the product of the first aspect is capable of increasing the viscoelastic ratio, R6, of skin with at least 10% over 8 weeks, preferably at least 15%, 20%, 25%, and most preferably at least 30%, when measured as defined herein

Other Ingredients

In a preferred embodiment, the product of the invention may also comprise other ingredients, preferably one or more active ingredient, preferably one or more pharmacologically active substance, and also preferably a water-soluble excipient, such as lactose.

In another preferred embodiment, the product of the invention may also comprise one or more enzyme(s), preferably a ligase, transferase, oxidoreductase, hydrolase, lyase, and/or an isomerase; more preferably an amylolytic enzyme, a lipolytic enzyme, a proteolytic enzyme, a cellulytic enzyme, an oxidoreductase or a plant cell-wall degrading enzyme, and more preferably an enzyme with an activity selected from the group consisting of aminopeptidase, amylase, amyloglucosidase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinase, peroxidase, phytase, phenoloxidase, polyphenoloxidase, protease, ribonuclease, transferase, transglutaminase, or xylanase.

Non-limiting examples of an active ingredient or pharmacologically active substance which may be used in the present invention include protein and/or peptide drugs, such as, human growth hormone, bovine growth hormone, porcine growth hormone, growth homorne releasing hormone/peptide, granulocyte-colony stimulating factor, granulocyte macrophage-colony stimulating factor, macrophage-colony stimulating factor, erythropoietin, bone morphogenic protein, interferon or derivative thereof, insulin or derivative thereof, atriopeptin-III, monoclonal antibody, tumor necrosis factor, macrophage activating factor, interleukin, tumor degenerating factor, insulin-like growth factor, epidermal growth factor, tissue plasminogen activator, factor IV, factor IIIV, and urokinase.

A water-soluble excipient my be included for the purpose of stabilizing the active ingredient(s), such excipient may include a protein, e.g., albumin or gelatin; an amino acid, such as glycine, alanine, glutamic acid, arginine. lysine and a salt thereof; carbohydrate such as glucose, lactose, xylose, galactose, fructose, maltose, saccharose, dextran, mannitol, sorbitol, trehalose and chondroitin sulphate; an inorganic salt such as phosphate; a surfactant such as TWEEN® (ICI), poly ethylene glycol, and a mixture thereof. The excipient or stabilizer may be used in an amount ranging from 0.001 to 99% by weight of the product.

Several aspects of the invention relate to various compositions and pharmaceutical comprising, among other constituents, an effective amount of the product as defined in the first aspect, and an active ingredient, preferably the active ingredient is a pharmacologically active agent; a pharmaceutically acceptable carrier, excipient or diluent, preferably a water-soluble excipient, and most preferably lactose.

In addition, aspects of the invention relate to articles comprising a product as defined in the first aspect or a composition as defined in the aspects and embodiments above, e.g., a cosmetic article, a sanitary article, a medical or surgical article. In a final aspect the invention relates to a medicament capsule or microcapsule comprising a product as defined in the first aspect or a composition as defined in other aspects and embodiments of the invention.

Methods of Using the Product or Composition

Various aspects of the invention relate to methods of performing treatment procedures, e.g., in the medical field, using a product of the first aspect, or using compositions of the invention.

One aspect relates to a method of performing procedures in ophtalmology, which comprises the use of a product as defined in the first aspect or a composition of the invention.

Another aspect relates to a method of performing procedures in the treatment of osteoarthritis, which comprises the use of a product as defined in the first aspect or a composition of the invention.

Yet another aspect relates to a method of performing procedures in the treatment of cancer, which comprises the use of a product as defined in the first aspect or a composition of the invention.

An aspect relates to a method of performing transdermal or dermal administration of a pharmacologically active agent, which comprises the use of a product as defined in the first aspect or a composition of the invention.

Another aspect relates to a method of performing dermal administration of a cosmetic, which comprises the use of a product or a composition of the invention.

### EXAMPLES

### Example 1

Various sodium hyaluronate (HA) fractions having different molecular weight were used. Table 1 summarizes the weight average molecular weight (Mw), the number average molecular weight (Mn), and polydispersities (I=Mw/Mn).

TABLE 1
Molecular properties of the different sodium hyaluronate used:

| Sodium hyaluronate | MW (Da) | Mn (Da) | I = Mw/Mn |
|---|---|---|---|
| HA 50 | 53 000 | 37 000 | 1.4 |
| HA 130 | 130 000 | 83 000 | 1.6 |
| HA 300 | 320 000 | 210 000 | 1.5 |

The efficacy tests of HA of well defined molecular weight in cosmetic formulations were performed using the following formulation (Table 2). A placebo cream was composed of the same ingredients but without HA.

TABLE 2
Composition of the cosmetic formulation denoted active cream:

| Ingredients | % w/w |
|---|---|
| Aqua | 72.25 |
| HA | 0.10 |
| Hydrogenated polydecene | 20.00 |
| Steareth-2 | 3.00 |
| Steareth-21 | 1.00 |
| Cetearyl alcohol | 1.50 |
| Phenoxyethanol, Methylparaben, Butylparaben, | 0.80 |
| Ethylparaben, Propylparaben, Isobutylparaben | |
| Diazolidinyl Urea | 0.25 |
| Disodium EDTA | 0.10 |

The aim of the study was to evaluate the anti-wrinkle efficacy of the cosmetic products after long-term use (1 month and 2 months) and to compare it to a placebo cream.

12 subjects applied the active cream and the placebo twice a day at home for 2 months. At the beginning of the study, after one month of treatment, and at the end of the study, instrumental measurements of skin hydration and elasticity were taken in the peri-ocular areas. A plastic replica was made of the skin surface in the same areas, and the micro-relief of skin stratum corneum was assessed with image analysis of the replica. Furthermore, digital photographs of the investigated areas were taken.

Method of Evaluation

The study was carried out in a bioclimatic room (24° C.; 50% rh). Volunteers were asked not to wash their faces and not to apply products on the areas involved in the test for at least 3 hours before performing the measurements. At the beginning of the study (T₀) instrumental evaluations of skin hydration, elasticity and roughness were carried out on the left and right peri-ocular areas, marked out in a reproducible way. Digital photographs of the same areas were taken, too.

The assessment was performed on the face, where one side of the face was treated with the active cream, and the other half side treated with a placebo cream as a control. The sides of application (left and right) of the two creams (active cream and placebo) on the faces were randomized, The subjects applied the two products on their face twice a day for two months.

After 1 month of treatment (T_{30d}) and at the end of the test (after 2 months of treatment, T_{60d}) the subjects returned to the laboratory to repeat the instrumental measurements and to take new digital images. The data obtained were then analysed and statistically compared.

Long Term Hydration

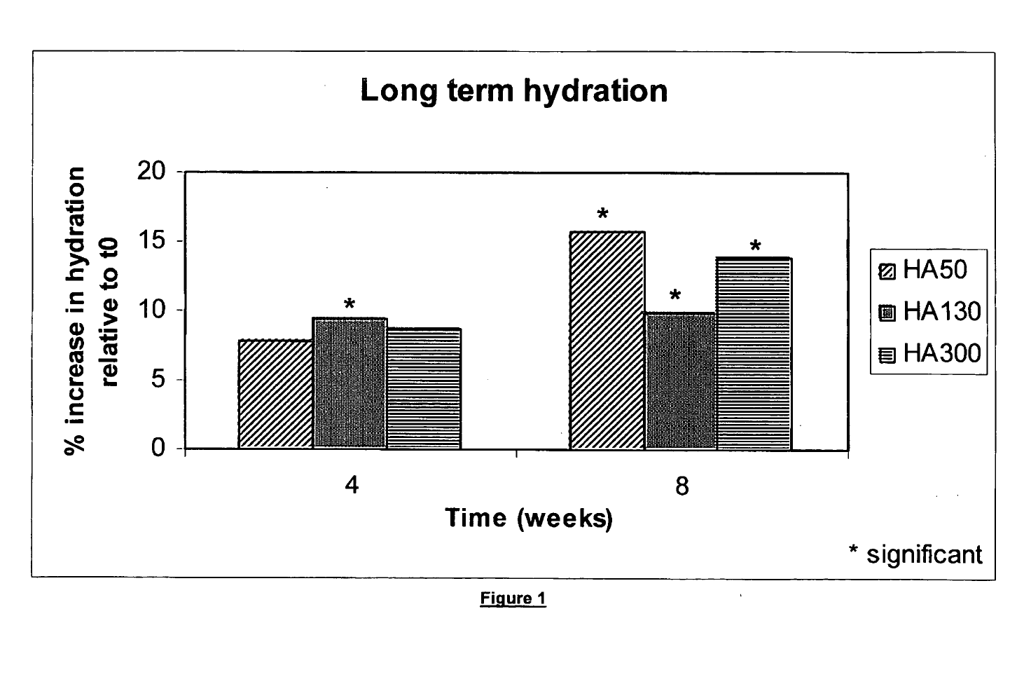

The evaluation of skin surface hydration was performed using Corneometry. Corneometry determines the capacitance of Stratum Corneum (SC) and thereby reflects the relative SC moisture. The measurement was performed using the Corneometer: Combi CM 825 (Courage & Khazaka). The results are shown in FIG. 1; a significant increase in hydration was clearly obtained with all molecular weights HA after 4 weeks and 8 weeks treatment,

Elasticity

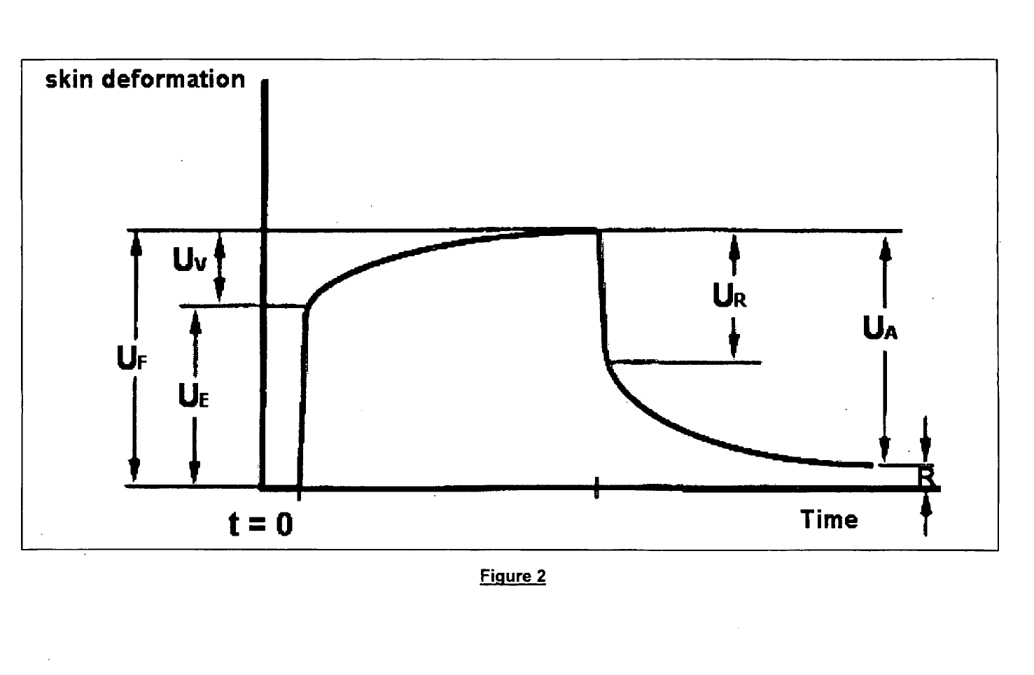

Skin elasticity was measured using the Cutometer SEM 575 (Courage & Khazaka). The Cutometer measures the vertical deformation of the skin, when sucked into the opening of a measuring probe. This method provides the deformation parameters relating to the skin elasticity as shown in FIG. 2, with the following paramenters:
UA/UF=overall elasticity of the skin (R2 parameter)
UV/UE=viscoelastic ratio (R6 parameter)
UA=Total deformation recovery at the end of the stress-off period
UF=Total extensibility of the skin
UV=Viscoelastic creep occurring after the elastic deformation
UE=Elastic deformation of the skin due to the application of stress.

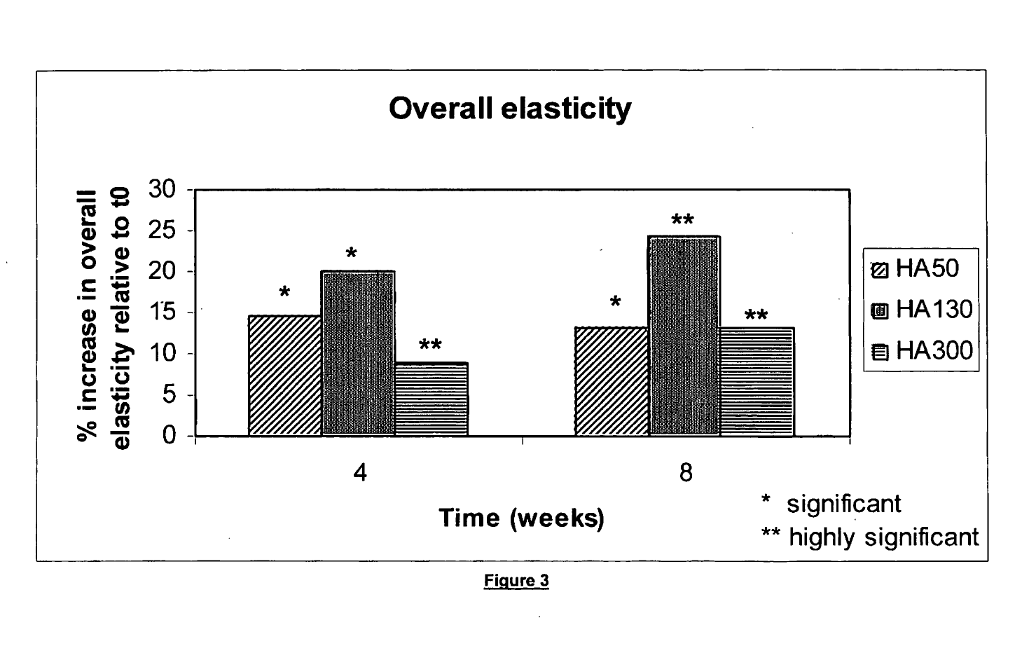

The measured overall elasticities, R2, after 8 weeks of application are shown in FIG. 3. A significant increase of the overall elasticity (R2) was clearly observed with all active creams. No significant difference was observed between the different molecular weight HA fractions.

Anti-wrinkle

The topography of the skin surface was evaluated by skin surface replicas and image analysis. The principle of the test is to obtain a negative imprint of the skin surface by applying a fast hardening synthetic polymer (SILFLO®—Flexico Ltd. UK.). This replica is then analyzed by image digitalization. From this image the standard roughness parameters Ra (mean roughness) and Rz (maximum roughness for deep wrinkles) were calculated.

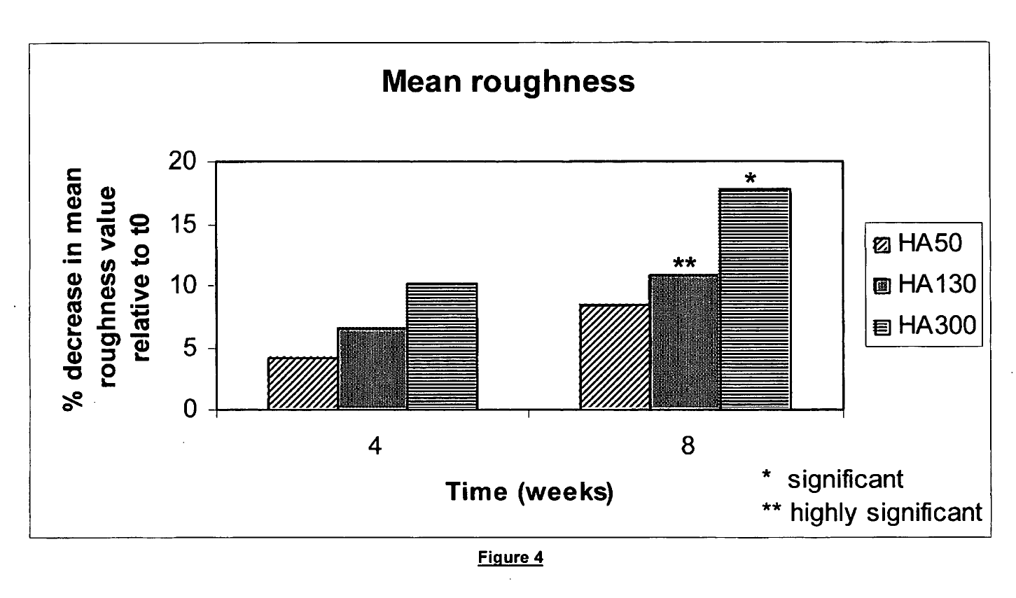

The mean roughness results are shown in FIG. 4. The mean roughness values decreased significantly after 4 and 8 weeks of application. The effect was more pronounced for the 300,000 Da MW fraction which accumulates preferentially at the surface of the skin.

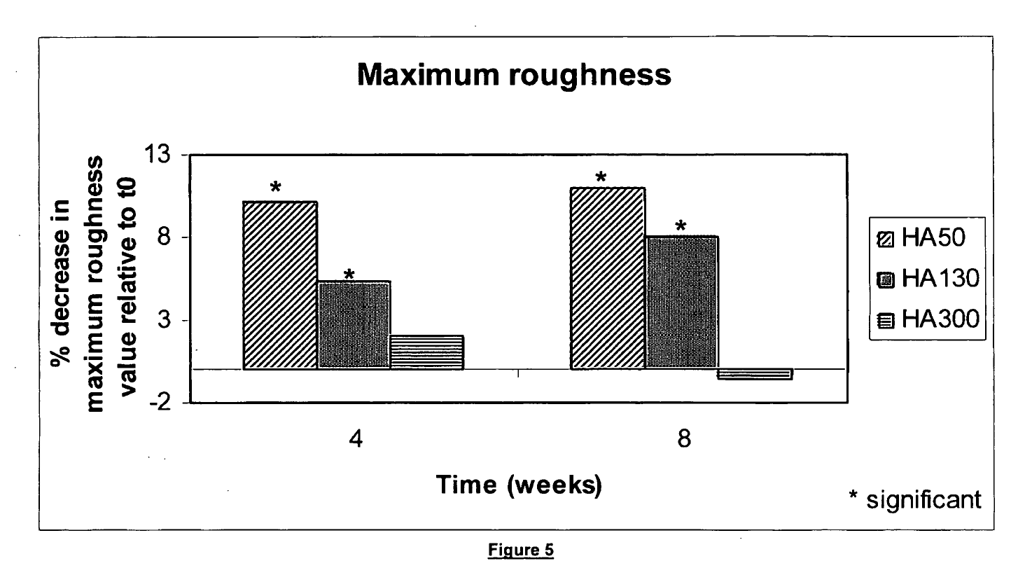

The max roughness results are shown in FIG. 5; these values also decreased significantly after 4 and 8 weeks of application, but only for the two lowest molecular weight fractions. This effect was significantly more pronounced for the very low molecular fraction HA of 50 kDa which is able to penetrate the skin.

1-34. (canceled)

35. A moisturizing, cosmetic, or anti-wrinkle product comprising at least two hyaluronic acid fractions, or salts thereof, wherein a fraction has an average molecular weight in the range of 8,000-100,000 Da, and a fraction has an average molecular weight in the range of 100,000-500,000 Da.

36. The product of claim 35, which comprises an inorganic salt of hyaluronic acid.

37. The product of claim 35, which is capable of increasing the skin hydration value with at least 3% over 8 weeks, when measured as defined herein.

38. The product of claim 35, which is capable of increasing overall skin elasticity, R2, with at least 4% over 8 weeks, when measured as defined herein.

39. The product of claim 35, which is capable of reducing the mean roughness value of skin with at least 5% over 8 weeks, when measured as defined herein.

40. The product of claim 35, which is capable of reducing the maximum roughness value of skin with at least 3% over 8 weeks, when measured as defined herein.

41. The product of claim 35, which is capable of increasing the viscoelastic ratio, R6, of skin with at least 10% over 8 weeks, when measured as defined herein.

42. The product of claim 35, Wherein the hyaluronic acid or salt thereof comprises esters of boric acid and/or a polymeric alpha hydroxy acid.

43. The product of claim 35, wherein the hyaluronic acid or salt thereof is fully or partially cross-linked with divinylsulfone (DVS).

44. The product of claim 35, which also comprises a water-soluble excipient.

45. A composition comprising a product as defined in claim 35 and an active ingredient.

46. The composition of claim 13, which also comprises a water-soluble excipient.

47. A pharmaceutical composition comprising an effective amount of a product as defined in claim 35 and a pharmaceutically acceptable carrier, excipient or diluent.

48. A pharmaceutical composition comprising an effective amount of a product as defined in claim 35 as a vehicle and a pharmacologically active agent.

49. A cosmetic article comprising as an active ingredient an effective amount of a product as defined in claim 35.

50. A sanitary, medical or surgical article comprising a product as defined in claim 35.

51. A medicament capsule or microcapsule comprising a product as defined in claim 35.

52. A method for the treatment of osteoarthritis, which comprises administering an effective amount of a product as defined in claim 35.

53. A method of treating osteoarthritis, comprising administering an effective amount of a product as defined in claim 35 to a mammal.

54. A method of treating a wound, comprising administering an effective amount of a product as defined in claim 35 to a mammal.
